(19) United States
(12) Patent Application Publication
(10) Pub. No.: US 2021/0330254 A1
(43) Pub. Date: Oct. 28, 2021

(54) NEUROLOGIC TOXICITY ASSESSEMENT METHODS AND SYSTEMS FOR DOCUMENTING CHANGES IN HANDWRITING

(71) Applicant: Janssen Biotech, Inc., Horsham, PA (US)

(72) Inventors: Kevin C. DE BRAGANCA, Chappaqua, NY (US); William DERAEDT, Schelle (BE); Erin C. LEE, Yardley, PA (US); Nikoletta LENDVAI, West Windsor, NJ (US); Maria Marquez de MONDELO, Belle Mead, NJ (US)

(21) Appl. No.: 17/235,974

(22) Filed: Apr. 21, 2021

Related U.S. Application Data
(60) Provisional application No. 63/015,425, filed on Apr. 24, 2020.
(60) Provisional application No. 63/090,094, filed on Oct. 9, 2020.

(51) Int. Cl. A61B 5/00; A61B 5/11; A61B 5/16; G16H 20/40

(52) U.S. Cl. CPC A61B 5/4848; A61B 5/1124; G16H 20/40; A61B 5/165; A61B 5/112

(57) ABSTRACT

Systems for assessing and documenting changes in handwriting neurologic toxicity adverse events associated with a medical treatment (e.g., immune effector cell therapy) comprise a handwriting neurotoxicity grading criteria that defines the symptoms of at least one adverse event including micrographia, dysgraphia and agraphia. The handwriting neurotoxicity grading criteria may be included in a system for assessing immune effector cell associated movement and neurocognitive disorder, that comprises grading criteria for one or more of the symptom domains: handwriting impairment, gait impairment, other movement abnormalities of extremities, impairment in ability to perform ADLs, and impairment in expression or motivation.

### CROSS-REFERENCE TO RELATED APPLICATIONS

This application claims the benefit of U.S. Provisional Application Ser. No. 63/015,425, filed 24 Apr. 2020 and U.S. Provisional Application Ser. No. 63/090,094, filed 9 Oct. 2020. The entire content of the aforementioned applications is incorporated herein by reference in its entirety.

### FIELD OF THE INVENTION

The present invention relates to methods and systems for assessing and documenting neurologic toxicity adverse events associated with a medical treatment or procedure and, in particular, to grading criteria for changes in handwriting adverse events relating to neurologic toxicity.

### BACKGROUND OF THE INVENTION

New prescription drugs must be approved by regulatory authorities, such as the Food and Drug Administration (FDA) in the United States, which, in part, requires that the manufacturer demonstrate the drug's safety and effectiveness under statutory and regulatory criteria. The drug development process begins with basic research in the laboratory, followed by preclinical development which commonly involves testing in animals to determine the drug's pharmacodynamics, pharmacokinetics, metabolism (ADME) and/or toxicology. The drug then proceeds to clinical testing in humans, at which point the regulatory authorities become involved.

In general, the clinical development of the drug involves three phases of clinical testing. Phase I clinical trials involve a relatively small group of individuals and evaluates the drug's safety and ideal dosage—e.g., to determine the highest dosage that may be safely administered without serious side effects. Phase II involves a larger group of individuals (e.g., <100) who have the condition that the drug is intended to treat, and evaluates the drug's effectiveness. Testing of a larger group of individuals may also reveal less common side effects. Phase III involves a group of at least several hundred individuals, and further evaluates the safety and effectiveness of the drug in comparison to existing methods of treatment—i.e. whether the new drug is at least as safe and effective as current standard treatments. As with Phases I and II, the individuals are monitored closely for side effects and treatment is stopped if they become too severe.

Clinical trials are initiated by a sponsor (e.g., a pharmaceutical company) who takes responsibility for and selects the principal investigator who conducts the clinical investigation. The sponsor must file an investigational new drug (IND) application with the FDA, that requires the sponsor to notify the FDA in a written report of any serious and unexpected adverse reactions that are or are reasonably suspected to be caused by the drug. An adverse reaction is generally defined as an adverse event (AE) or any unfavorable and unintended sign, symptom, or disease that is associated with the drug or medical treatment, whether or not adverse event is considered related to the drug or medical treatment.

To promote consistency in the assessment and reporting of adverse events within a clinical trial and to facilitate comparison across different clinical studies, it is useful to have a standard terminology and scale that provides a framework for documenting the severity of adverse events. For example, the National Cancer Institute has published the Common Terminology Criteria for Adverse Events (CTCAE), which provides grading criteria for assessing the severity of organ toxicity relating to cancer therapy. The CTCAE comprises an extensive list of terms for adverse events that are commonly encountered during oncology-related clinical trials, and defines grades from 1-5 that describe symptoms of increasing severity associated with the adverse events. In general, Grade 1 corresponds to asymptomatic or mild symptoms, Grade 2 moderate, Grade 3 severe or medically significant requiring hospitalization, Grade 4 life-threatening, and Grade 5 death.

The CTCAE grading criteria provide standardization and consistency in defining and reporting treatment-related toxicity within a clinical trial and across different clinical studies. However, the CTCAE criteria provide guidance for a subset of toxicities and may become limited as new drugs and medical treatments are developed, such as cellular therapies which may involve new toxicities that physicians must learn to monitor and treat appropriately.

For example, immune effector cell therapies include autologous chimeric antigen receptor (CAR) T-cell therapy, which is a new type of treatment that involves the harvesting and genetic engineering of a patient's own T cells to recognize specific antigens on the surface of tumor cells. The genetically engineered T cells are reintroduced to the patient, where they bind specifically to the tumor cells and initiate the immune response to kill the tumor cells. The side effect profile of these CAR T-cell therapies is different from the classic treatments such as conventional chemotherapies or even more recently developed therapies such as treatment with monoclonal antibodies. Common toxicities that are associated with CAR T-cell therapy include cytokine release syndrome (CRS) and neurotoxicity. Grading criteria for CRS are provided by the CTCAE and have also been developed by the American Society for Transplantation and Cellular Therapy (ASTCT). However, there is a lack of adequate tools for describing and grading adverse events relating to neurotoxicity, where the symptoms may include headache, confusion, delirium, language disturbance (e.g., aphasia), seizures and, in rare cases, acute cerebral edema.

The ASTCT has developed criteria for Immune Effector Cell-Associated Neurotoxicity Syndrome (ICANS), that includes a 10-point grading tool for Immune Effector Cell-Associated Encephalopathy (ICE). The ICE tool comprises a weighted sum of five elements: orientation (4 points), naming (3 points), following commands (1 point), writing (1 point) and attention (1 point). However, it is possible that further developments in CAR T-cell therapy and other types of treatment may present unique toxicities, where the ICE tool does not provide appropriate weighting and/or sufficient detail to effectively describe and report associated adverse events. For example, changes in handwriting may be of greater significance in (early) detection and evaluation of CAR T-cell related neurotoxicity, yet no common criteria exist for assessing and documenting these types of adverse events. Thus, there is a need for additional and standardized grading criteria for defining and reporting adverse events relating to immune effector cell therapy associated neurotoxicity.

### BRIEF SUMMARY OF THE INVENTION

The invention satisfies these needs and other needs by providing an assessment and grading system for the assessment of changes in handwriting associated with immune effector cell therapy in patients.

In one embodiment, a system for assessing neurologic toxicity in a subject associated with a medical treatment comprises at least two grading criteria selected from the group consisting of: handwriting impairment, gait impairment, other movement abnormalities of extremities, impairment in the ability to perform self-care activities of daily living, and impairment in expression or motivation. Each grading criteria define the symptoms of at least one adverse event. The grading criteria are stored in a non-transitory computer-readable medium.

In one embodiment, a system for assessing neurologic toxicity in a subject associated with a medical treatment comprises: a grading criteria for handwriting impairment that defines the symptoms of at least one adverse event selected from the group consisting of: micrographia, dysgraphia, and agraphia; a grading criteria for gait impairment that defines the symptoms of at least one adverse event selected from the group consisting of: Parkinsonian gait, gait disturbance, gait inability, and posture abnormal; a grading criteria for other movement abnormalities of extremities that defines the symptoms of at least one adverse event selected from the group consisting of: tremor, muscle rigidity, cogwheel rigidity, restlessness, akathisia, bradykinesia, hypokinesia, coordination abnormal, ataxia, and balance disorder; a grading criteria for impairment in the ability to perform self-care activities of daily living that defines the symptoms of at least one adverse event selected from the group consisting of: impaired self-care, dysphagia, dyspraxia, and apraxia; and a grading criteria for impairment in expression or motivation that defines the symptoms of at least one adverse event selected from the group consisting of: flat affect, reduced facial expression, amimia, apathy, indifference, communication disorder, slow speech, and dysarthria. The grading criteria are stored in a non-transitory computer-readable medium. In a further embodiment, the grading criteria for handwriting impairment defines the symptoms of at least two grades of severity of the adverse event, comprising: a first grade defined as mildly smaller or slower writing or difficulty in completing a writing task from baseline, but most words are legible; and a second grade defined as moderate to severely smaller or slower writing or difficulty in completing task from baseline, and most words are illegible. The grading criteria for gait impairment defines the symptoms of at least four grades of severity of the adverse event, comprising: a first grade defined as a slower walk that requires no assistance, and no festination or propulsion; a second grade defined as walking with difficulty that requires little assistance; a third grade defined as a severe gait disturbance that requires assistance; and a fourth grade defined as an inability to walk even with assistance. The grading criteria for other movement abnormalities of extremities defines the symptoms of at least three grades of severity of the adverse event, comprising: a first grade defined as mild symptoms of tremors at rest, rigidity, slowing of movements, restlessness, impaired coordination, or impaired balance; a second grade defined as moderate symptoms of tremors at rest, rigidity, slowing of movements, restlessness, impaired coordination, or impaired balance; and a third grade defined as severe symptoms of tremors at rest, rigidity, slowing of movements, restlessness, impaired coordination, or impaired balance. The grading criteria for impairment in the ability to perform self-care activities of daily living defines the symptoms of at least four grades of severity of the adverse event, comprising: a first grade defined as performing self-care activities of daily living somewhat slower than baseline, but no help needed for self-care; a second grade defined as requiring occasional assistance with self-care activities of daily living; a third grade defined as requiring considerable help to perform self-care activities of daily living, but can do some things alone; and a fourth grade defined as disabled and requiring full care. The grading criteria for impairment in expression or motivation defines the symptoms of at least two grades of severity of the adverse event, comprising: a first grade defined as mildly flat affect, reduced facial expression or emotional response, less communicative, slower or mumbled speech, or disinterest in non-routine activities; and a second grade defined as moderate to severely flat affect, lack of facial expression or emotional response, few word answers, monotonic or slurred speech, or disinterest in routine activities.

In one embodiment, a method of assessing neurologic toxicity in a subject associated with a medical treatment comprises the step of determining a toxicity grade according to at least one grading criteria selected from the group consisting of: a grading criteria for handwriting impairment, a grading criteria for gait impairment; a grading criteria for other movement abnormalities of extremities; a grading criteria for impairment in the ability to perform self-care activities of daily living; and a grading criteria for impairment in expression or motivation.

### BRIEF DESCRIPTION OF THE DRAWINGS

The foregoing summary, as well as the following detailed description of preferred embodiments of the present application, will be better understood when read in conjunction with the appended drawings. It should be understood, however, that the application is not limited to the precise embodiments shown in the drawings.

FIG. 1 is a table comprising grading criteria embodying a system for assessing and grading handwriting neurologic toxicity in an individual.

FIG. 2 shows a handwriting log for recording, assessing and grading handwriting neurologic toxicity in an individual, for use with the system and grading criteria of FIG. 1.

FIGS. 3A and 3B comprise a table comprising grading criteria embodying a system for assessing and grading neurologic toxicity symptoms in an individual, that are associated with immune effector cell therapy.

### DETAILED DESCRIPTION OF THE INVENTION

Methods and systems are described for documenting and assessing adverse events relating to neurologic toxicity in clinical trials for new drugs and medical treatments. Common symptoms associated with neurologic toxicity include headache, confusion, delirium, language disturbance (e.g., aphasia), seizures and, in rare cases, acute cerebral edema. Other common symptoms of immune effector cell associated neurotoxicity include changes in handwriting. The CTCAE (version 5.0) does not define any grading criteria for handwriting adverse events, nor are there any other standardized toxicity grading criteria available to assess changes in handwriting.

The International Council for Harmonisation of Technical Requirements for Pharmaceuticals for Human Use (ICH) has developed the Medical Dictionary for Regulatory Activities (MedDRA), which provides standardized medical terminology for sharing regulatory information relating to medical products for use in humans. MedDRA has a hierarchical structure, in which higher level terms comprise groups of related lower level terms of increasing specificity. The lowest, most specific level consists of Lowest Level Terms, which are terms that reflect terminology used in practice—e.g., “feeling queasy”. The next level consist of Preferred Terms, which are terms for medical concepts that represent a group of one or more related Lowest Level Terms—e.g., “nausea”. Higher levels further comprise terms which represent groups of lower level terms that are related by anatomy, pathology, physiology, etiology or function.

The MedDRA Preferred Terms for changes in handwriting are: micrographia, dysgraphia, and agraphia. Micrographia is a disorder characterized by abnormally small or cramped handwriting, or the progression to increasingly small handwriting. Dysgraphia is a transcription disability characterized by writing that is illegible, requires extreme effort or unreasonable amount of time to accomplish and/or exhibits poor spelling. Agraphia is the loss of ability to communicate by writing, characterized by the inability to write meaningful words and/or writing that is slow, requires excessive effort and is distorted or incomplete.

In one embodiment, a system for assessing and documenting adverse events relating to neurologic toxicity in a subject comprises grading criteria for one or more handwriting adverse events. In an embodiment, the adverse events correspond to one or more MedDRA Lowest Level Terms and/or Preferred Terms, such as the Preferred Terms micrographia, dysgraphia and agraphia. In a preferred embodiment, the handwriting grading criteria defines at least two grades of severity of symptoms for an adverse event. The grading criteria preferably correspond to the CTCAE grading system—i.e. asymptomatic or mild (Grade 1), and moderate (Grade 2). Additional grades for severity of the adverse events may be provided. However, higher toxicity grades under the CTCAE guidelines are not applicable to the assessment of changes in handwriting—i.e. are defined as adverse events that involve hospitalization (Grade 3), are life-threatening (Grade 4), are disabling or result in death (Grade 5).

In an embodiment, the grading criteria define at least two grades of severity of symptoms for micrographia. Grade 1 is defined as handwriting exhibiting a decrease in the size of the letters or spacing of the words of the sentence of less than 50% in comparison to a baseline handwriting sample. Grade 2 is defined as handwriting exhibiting a decrease of 50% or more in size or spacing in comparison to the baseline.

In an embodiment, the grading criteria define at least two grades of severity of symptoms for dysgraphia. Grade 1 is defined as handwriting of a predetermined sentence in comparison to a baseline handwriting sample that exhibits symptoms including mildly slower writing, impaired straightness of line, difficulty in completing the sentence, and most but not all words of the sentence are legible. Grade 2 is defined as handwriting in comparison the baseline that exhibits the symptoms of Grade 1, but with moderate to severely slower writing, and most words of the sentence are illegible.

In an embodiment, the grading criteria define at least two grades of severity of symptoms for agraphia. Grade 1 is defined as handwriting of a predetermined sentence in comparison to a baseline handwriting sample that exhibits symptoms including the inability to write the same number of words of the sentence as the baseline but at least 3 words. Grade 2 is defined as handwriting in comparison to the baseline that exhibits the symptoms of Grade 1, but with the inability to write more than 2 words.

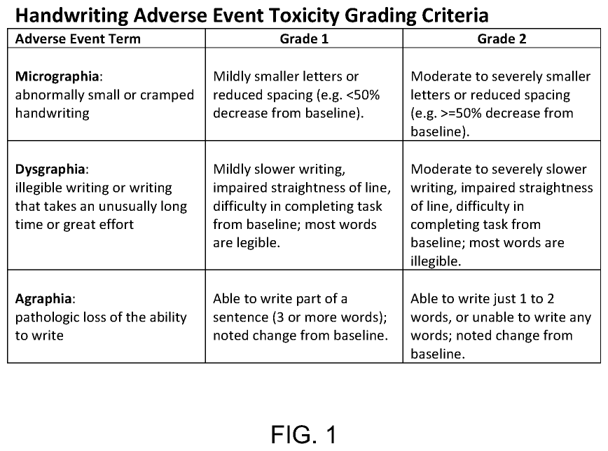

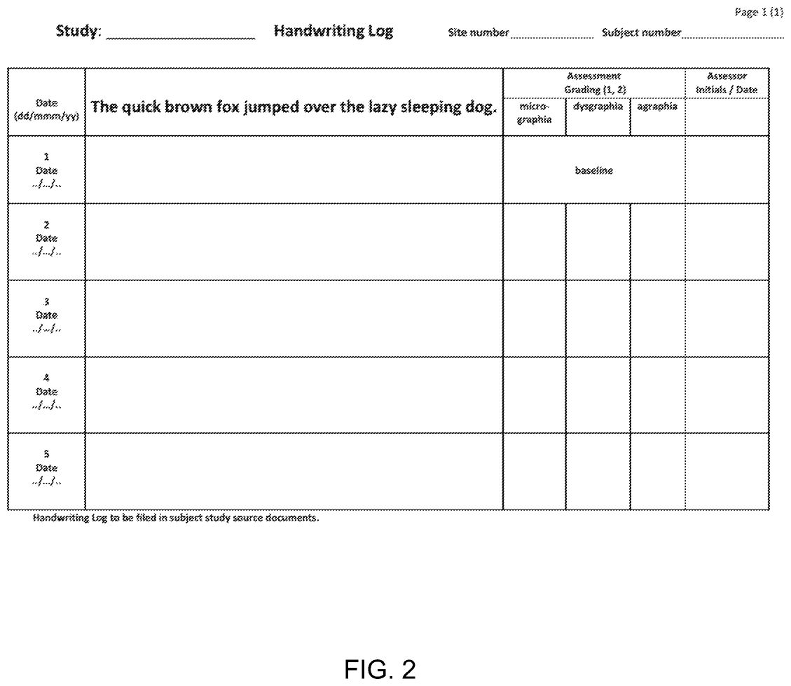

FIG. 1 shows an embodiment of a handwriting neurotoxicity grading criteria in the form of a reference guide for use in assessing the changes in a subject's handwriting. Preferably, a copy of the handwriting neurotoxicity grading criteria is stored in digital form, such as in a non-transitory computer-readable medium. The grading criteria provide consistency in assessing and reporting handwriting adverse events in a clinical trial and across different clinical trial groups. The system may also include a form for recording adverse events, such as a handwriting log for taking samples of the subject's handwriting over time. Preferably, a copy of the handwriting log is stored in digital form, such as in a non-transitory computer-readable medium. An embodiment of a handwriting log for use with the handwriting grading criteria is shown in FIG. 2. In a preferred embodiment, the form provides for recording: (1) an identifier of the clinical trial, (2) an identifier of the test subject, (3) a baseline sample and one or more additional samples of the subject's handwriting, (4) the date of the sample, (5) the assessment grade (Grade 1 or 2) for the three adverse event categories (micrographia, dysgraphia, agraphia), and (6) the date of the assessment and an identifier for the person conducting the assessment.

The system may also comprise a protocol and instructions for conducting the handwriting assessment to be performed by the principal investigator of the clinical trial, or his/her designee. The test subject is asked to provide a handwriting sample of a test phrase, which may be spontaneous or predetermined. In one embodiment, the test phrase is a relatively short predetermined standard phrase comprised of common words. In a preferred embodiment, the standard phrase comprises about 10 words, and more preferably includes all or most of the letters of the alphabet. An example of a suitable standard phrase is, “The quick brown fox jumped over the lazy sleeping dog.” In an embodiment, the standard phrase may be printed on the recording form for copying by the test subject, as shown in FIG. 2.

In practice, an initial or baseline handwriting sample is taken at the start of the clinical trial, prior to administering the drug or medical treatment. Subsequent handwriting samples that are taken over the course of the clinical trial may be compared to the baseline for the assessment of any changes in the subject's handwriting. The subject's handwriting samples are preferably taken at the beginning of each visit, before any interventions have been performed. The subject is provided with a writing instrument with which he/she is comfortable, and is placed in a comfortable position to write (e.g., seated at a table or sitting up in a bed using a bedside table). If the handwriting samples are recorded in a log, the prior handwriting samples are covered or otherwise hidden from the subject's view during the current assessment. The same brand and type of writing instrument is preferably used for all subsequent writing samples. Writing instruments that are erasable (e.g., pencils), or that may require some skill or familiarity to use comfortably (e.g., fountain pens, or pens with large grips) are generally not recommended. In one embodiment, the writing instrument is a black or blue ballpoint pen. Preferably, a copy of the handwriting sample(s) is stored in digital form, such as in a non-transitory computer-readable medium.

The individual (examiner) performing the assessment of the handwriting sample should observe the subject while writing the test phrase, to allow for a complete assessment of changes in writing ease or difficulty from baseline—e.g., (greater) pauses between words, increased number of pen lifts, or breaks in flow in the middle of words. The assessment of the handwriting samples is preferably administered by the same individual to ensure consistency of grading and reporting. The handwriting samples are assessed for adverse events in the categories of micrographia, dysgraphia and agraphia, pursuant to the grading criteria provided in the reference guide. The assessment of dysgraphia may include a subjective component that is based on the examiner's recall, the subject's self-report (e.g. the subject notes writing is slower and more difficult to perform) and/or the observations of another person (e.g., the subject's caregiver).

In the event that the subject has a condition that would prevent effective comparison of the handwriting sample with the baseline (e.g., hand injury, peripheral neuropathy, muscle cramps, systemic condition like the flu, or depressed level of consciousness), the examiner should document the condition on the log in connection with the handwriting sample. The principal investigator of the clinical trial may choose to omit such writing samples from assessment.

Preferably, a copy of the graded handwriting sample(s) is stored in digital form, such as in a non-transitory computer-readable medium. The digital copies of the handwriting neurotoxicity grading criteria, handwriting log, handwriting samples and/or graded handwriting samples may each be stored in a separate non-transitory computer-readable medium, or more preferably are stored in the same non-transitory computer-readable medium.

The detection of handwriting neurotoxicity may prompt different actions depending on the severity of the adverse events (e.g., Grade 1 or Grade 2), and the nature of the clinical trial (e.g., the type of drug or other medical treatment, and the medical condition being investigated). For example, the detection of handwriting neurotoxicity may suggest the need for close observation of the subject, further work up of the subject and/or assessment of other neurotoxicity symptoms. Alternatively, the detection of handwriting neurotoxicity may indicate the need for intervention and/or stopping the treatment.

In one embodiment, the above-described system and methods for documenting and assessing adverse events using the handwriting neurotoxicity grading criteria, is applied to subjects that are under medical treatment for malignancy. In a preferred embodiment, the medical treatment is immune effector cell therapy and more preferably, CAR T-cell therapy targeting B-cell Maturation Antigen (BCMA). Common neurologic toxicities associated with CAR T-cell therapy include aphasia and disorientation. It has been found that anti-BCMA CAR T-cell therapy is associated with neurologic toxicities that may be unique and have not yet been publicly reported in other existing CAR-T products. These novel toxicities appear a little later than the typical CAR-T associated neurotoxicity (i.e. after CRS resolution), and present atypically with neurocognitive and/or motor symptoms. Therefore, it is desirable to provide standardized grading criteria for defining and reporting additional neurotoxicity adverse events, including changes in handwriting. Furthermore, the detection of handwriting adverse events such as micrographia, dysgraphia, and agraphia may provide an early indication of patients that are at potential risk for other, more serious neurotoxicities. Early detection would allow these at-risk patients to be monitored closely for early treatment, and may be key to a more optimal outcome.

Subjects in clinical trials or other clinical studies involving immune effector cell therapy are preferably assessed for handwriting adverse events using the handwriting neurotoxicity grading criteria. Grade 1 or Grade 2 micrographia, dysgraphia and/or agraphia adverse events should be reported promptly when these changes in handwriting are detected, along with other immune effector cell associated neurotoxicities (e.g., ICANS or other neurotoxicity). This information should prompt further assessment for other neurotoxicity symptoms, further workup, as well as the potential initiation of interventions.

The grading criteria for handwriting adverse events may be incorporated in a system for assessing and grading neurologic toxicity symptoms that are associated with immune effector cell therapies, such as CAR T-cell therapy, which may not be recognized or easily documented using conventional CTCAE guidelines. In one embodiment, a system for assessing and documenting adverse events relating to neurologic toxicity in a subject comprises grading criteria for one or more symptoms that are associated with Immune effector Cell Associated Movement And Neurocognitive Disorder (ICAMAND). The MedDRA Preferred Term that generally comprises such movement and neurocognitive disorders is Parkinsonism, which is defined by impaired motor symptoms, cognitive impairment, and/or personality changes. Impaired motor symptoms are a general slowing of movements and rigidity. Loss of gross motor control is characterized by difficulty walking, poor balance, restlessness, and changes in gait including shuffling, festination, or propulsion. Loss of fine motor control is characterized by difficulty writing or performing self-care activities of daily living (ADLs), impaired coordination, and tremors. Examples of ADLs include, but are not limited to, dressing, feeding, taking medication, and hygiene. Cognitive impairment is general mental slowness, characterized by short-term memory impairment, forgetfulness, difficulty word finding, slow speech, poor concentration, and difficulty problem solving or learning new things. Personality changes are commonly characterized by flat affect and diminished emotional response, which may include apathy, lethargy, indifference, less communicative, loss of initiative or interest, and diminished facial expression or emotional response.

The ICAMAND grading criteria defines the symptoms of adverse events in at least one, and preferably at least two of the Symptom Domains comprising: (1) handwriting impairment, (2) gait impairment, (3) other movement abnormalities of extremities, (4) impairment in ability to perform ADLs, and (5) impairment in expression or motivation. Each Symptom Domain comprises at least one adverse event that is reported consistent with the applicable MedDRA Lowest Level Terms and/or Preferred Terms. The grading criteria define the symptoms of at least two grades of severity of the adverse events, which are assessed as a change from a baseline. In one embodiment, the grading criteria reflect the CTCAE grading system, and comprise: (Grade 1) mild, intervention not indicated; (Grade 2) moderate, noninvasive intervention indicated; (Grade 3) severe or medically significant but not immediately life-threatening, including disabling and/or hospitalization or prolongation of hospitalization indicated; (Grade 4) life-threatening or profound disability, including intervention indicated to prevent permanent impairment, persistent disability, or death, hospitalization or prolongation of hospitalization indicated, including skilled nursing facility. CTCAE Grade 5, which corresponds to death related to an adverse event, is generally not applicable to the ICAMAND grading criteria.

The grading criteria for handwriting impairment are discussed above. In an embodiment, adverse events relating to handwriting impairment are reported consistent with at least one of the MedDRA Preferred Terms: micrographia, dysgraphia, and agraphia. In a preferred embodiment, the grading criteria comprise two grades handwriting impairment. Grade 1 is defined as mildly smaller or slower writing or difficulty in completing a writing task from baseline, but most words are legible. Grade 2 is defined as moderate to severely smaller or slower writing or difficulty in completing task from baseline, and most words are illegible. Grades 3 and 4 are not applicable. The assessment of changes in handwriting may be performed using a form for recording a subject's handwriting sample of a standard phrase, as discussed above. In an embodiment, the standard phrase may be omitted from the recording form. Although suitable for evaluation of handwriting, copying may not allow the assessment of adverse events in other Symptom Domains, such as communication disorder.

In an embodiment, adverse events relating to gait impairment are reported consistent with at least one of the MedDRA Preferred Terms: Parkinsonian gait, gait disturbance, gait inability, and posture abnormal. In a preferred embodiment, the grading criteria comprise four grades of severity of symptoms for gait impairment. Grade 1 is defined as a slower walk that requires no assistance; and may include arm swinging, a shuffling gait, or a stooped posture, but no festination or propulsion. Grade 2 is defined as walking with difficulty that requires little assistance; and may include some festination or propulsion with a shuffling gait or stopped posture. Grade 3 is defined as a severe gait disturbance that requires assistance. Grade 4 is defined as an inability to walk even with assistance—e.g., the subject is bedridden or chairbound.

In an embodiment, adverse events relating to other movement abnormalities of extremities comprise movement abnormalities that are not included in Symptom Domains (1) and (2)—i.e. exclude adverse events relating to handwriting impairment and gait impairment. Adverse events relating to other movement abnormalities of extremities are reported consistent with at least one of the MedDRA Preferred Terms: tremor, muscle rigidity, cogwheel rigidity, restlessness, akathisia, bradykinesia, hypokinesia, coordination abnormal, ataxia, and balance disorder. Cogwheel rigidity is a type of rigidity in which the muscles respond with cogwheel like jerks to the use of force in bending a limb. Akathisia is a movement disorder that is characterized by difficulty in staying still. Hypokinesia is another type of movement disorder that refers to little movement. Bradykinesia describes slow movements and reflexes and is often a symptom of a medical condition (e.g., Parkinson's disease). Ataxia is a symptom of nervous system dysfunction that is characterized by the lack of muscle control or coordination of voluntary movements. A subject who suffers from ataxia may display symptoms such as slurred speech, stumbling, falling, and incoordination. Balance disorder is a condition that makes one feel unsteady or dizzy.

In a preferred embodiment, the grading criteria comprise three grades of severity of symptoms for other movement abnormalities of extremities. Grade 1 is defined as mild symptoms of tremors at rest, rigidity, slowing of movements, restlessness, impaired coordination, or impaired balance. Grade 2 is defined as moderate symptoms of tremors at rest, rigidity, slowing of movements, restlessness, impaired coordination, or impaired balance. Grade 3 is defined as severe symptoms of tremors at rest, rigidity, slowing of movements, restlessness, impaired coordination, or impaired balance. Grade 4 is not applicable.

In an embodiment, adverse events relating to impairment in ability to perform ADLs are reported consistent with at least one of the MedDRA Preferred Terms: impaired self-care, dysphagia, dyspraxia, and apraxia. Dysphagia is a disorder characterized by difficulty in swallowing that can be associated with pain. Dyspraxia is a motor learning disability that includes difficulty with fine motor skills, gross motor skills, motor planning, and coordination. Apraxia is a neurological disorder that is characterized by the inability to perform familiar movements on command, despite the fact that the command is understood and there is a willingness to perform the movement.

In a preferred embodiment, the grading criteria comprise four grades of severity of symptoms for impairment in ability to perform self-care ADLs. Grade 1 is defined as performing self-care ADLs somewhat slower than baseline, but no help needed for self-care. Grade 2 is defined as requiring occasional assistance with self-care ADLs, including buttoning, cutting food, brushing teeth, combing hair, and/or self-feeding. Grade 3 is defined as requiring considerable help to perform self-care ADLs, but can do some things alone, and may include notable difficulty with self-feeding or swallowing. Grade 4 is defined as disabled and requiring full care.

In an embodiment, adverse events relating to impairment in expression or motivation are reported consistent with at least one of the MedDRA Preferred Terms: flat affect, reduced facial expression, amimia, apathy, indifference, communication disorder, slow speech, and dysarthria. Amimia is an inability to use or understand hand gestures or body language. Apathy and indifference refer to a lack of interest in activities and/or social interactions. Communication disorder is a neurocognitive disorder that is characterized by impairments in sending, receiving, processing, or understanding verbal, nonverbal, or graphic language, speech, and/or communication. Dysarthria is a motor speech disorder in which the muscles that are used to produce speech are damaged, paralyzed or weakened.

In a preferred embodiment, the grading criteria define two grades of severity of symptoms for impairment in expression or motivation. Grade 1 is defined as mildly flat affect, reduced facial expression or emotional response, less communicative, slower or mumbled speech, or disinterest in non-routine activities. Grade 2 is defined as moderate to severely flat affect, lack of facial expression or emotional response, few word (terse) answers, monotonic or slurred speech, or disinterest in routine activities. Grades 3 and 4 are not applicable.

The adverse events corresponding to the Symptom Domains are not limited to MedDRA Preferred Terms. In alternative embodiments, the adverse events may be reported consistent with Lowest Level Terms and/or a combination of Preferred Terms and Lowest Level Terms.

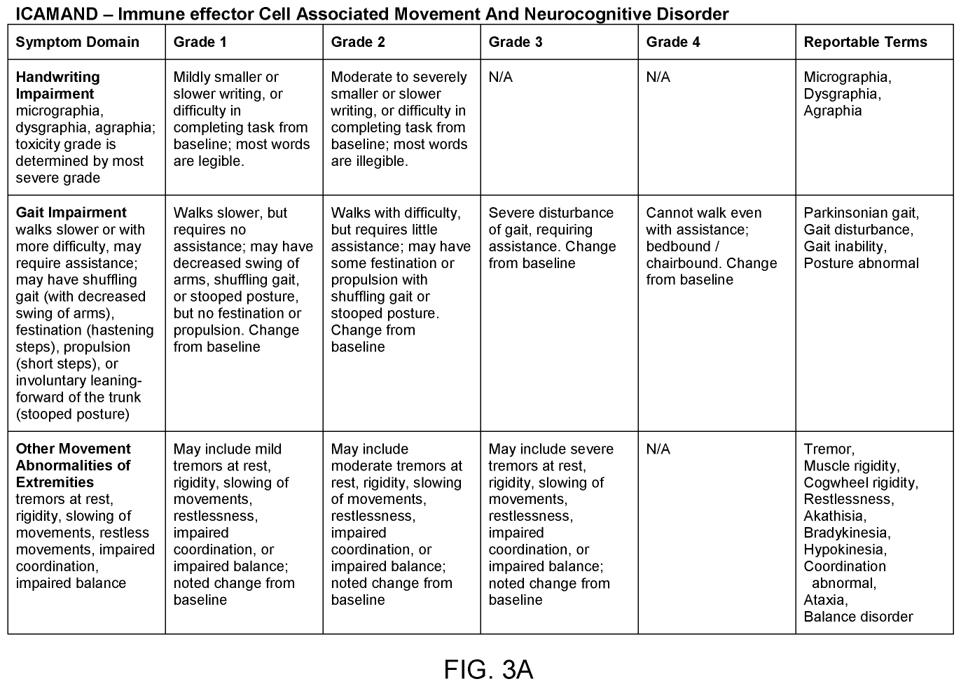

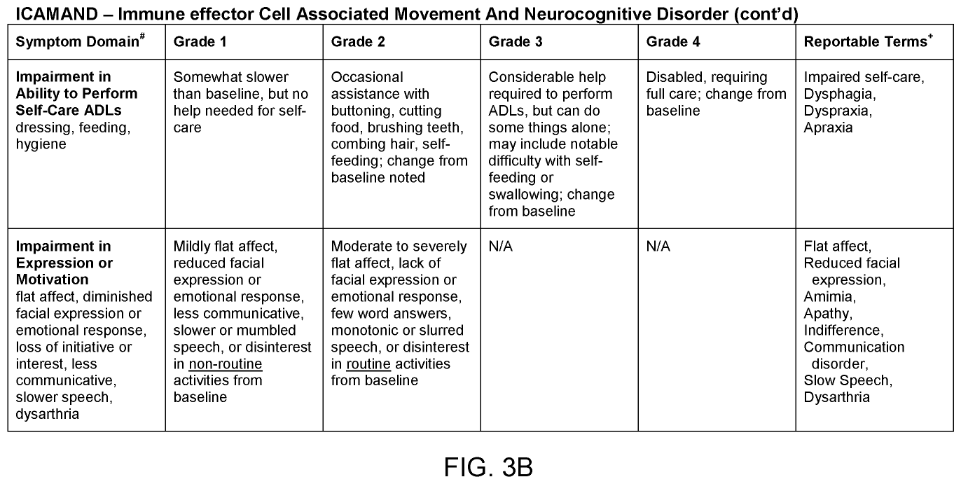

FIG. 3 shows an embodiment of ICAMAND grading criteria in the form of a reference guide. A copy of the ICAMAND grading criteria may be stored in digital form, such as in a non-transitory computer-readable medium. The system may also include a form or log for recording adverse events. A copy of the log may also be stored in digital form, such as in a non-transitory computer-readable medium.

Impairments should be noted after CRS or ICANS resolution, in the absence of other clear etiologies for neurocognitive decline—e.g. fever, infection, neoplasm, cerebrovascular disease, pre-existing neurological disease, substance abuse, etc. In one embodiment, neurologic toxicity in a subject is assessed by determining a toxicity grade according to one or more Symptom Domain grading criteria. An overall ICAMAND Toxicity Grade is determined as the toxicity grade of the most severely impaired Symptom Domain. In a preferred embodiment, a diagnosis of Immune effector Cell Associated Movement And Neurocognitive Disorder preferably requires that at least 2 domains are impacted and symptoms are present for at least 1 week.

Neurology consultation and evaluation should be considered at the first sign of neurotoxicity. In practice, cognitive impairment and personality changes are commonly seen with ICAMAND, and can present with an insidious onset. Cognitive assessment and evaluation should be considered for subjects presenting with changes in cognition and observed symptoms reported. In one embodiment, all observed symptoms are reported, including adverse events that are not represented under the MedDRA Preferred Terms. Examples of reportable symptoms include cognitive disorder, short-term memory impairment, slowness of thought, forgetfulness, mental impairment, word finding difficulty, poor concentration, disturbance in attention, impaired problem solving, personality change, social withdrawal, lethargy, and sleep disorders, including sleep movement disorders.

### Example 1: Case Study of Neurotixicity Patient Detected Using the Handwriting Assessment Tool

One case of CAR-T Associated Movement and Neurocognitive (CAMAND) was identified through the use of this handwriting tool (the only new CAMAND case reported for the study product since the handwriting tool was implemented in clinical trials). The patient was pre-identified to be at risk for CAMAND due to baseline risk factors (male gender, high disease burden, limited response to bridging therapy) and notable toxicities associated with increased risk post CAR-T infusion (Grade 3 CRS). The subject returned home from the hospital approximately 28 days after CAR-T infusion, after which the patient's spouse observed daytime sleepiness, fatigue, apathy, and some changes in handwriting at home between Days 28 and the study visit on Day 56. At the Day 56 study visit, the use of the handwriting tool assisted the investigator in diagnosing Grade 1 micrographia, which prompted further neurological evaluation per study protocol. During this visit, changes in balance and coordination, bradykinesia, rigidity, and Parkinsonian gait and stance were noted, in addition to personality changes such as flat affect, masked facies, less communicativeness, and apathy. This case exemplifies that handwriting monitoring and early detection of changes in handwriting-via this tool prompts further neurological assessment and enabled efficient identification of CAR-T Associated Movement and Neurocognitive Disorder.

While particular embodiments of the present disclosure have been illustrated and described, it would be obvious to those skilled in the art that various other changes and modifications can be made without departing from the spirit and scope of the present disclosure. It is therefore intended to cover in the appended claims all such changes and modifications that are within the scope of this disclosure.

I/We claim:

1. A system for assessing neurologic toxicity in a subject associated with a medical treatment, comprising:
at least two grading criteria selected from the group consisting of: handwriting impairment, gait impairment, other movement abnormalities of extremities, impairment in the ability to perform self-care activities of daily living, and impairment in expression or motivation;
wherein each grading criteria define the symptoms of at least one adverse event, and wherein the grading criteria are stored in a non-transitory computer-readable medium.

2. The system of claim 1, comprising grading criteria for handwriting impairment, that defines the symptoms of at least one adverse event selected from the group consisting of: micrographia, dysgraphia, and agraphia.

3. The system of claim 2, wherein the grading criteria for handwriting impairment defines the symptoms of at least two grades of severity of the adverse event, comprising:
a first grade defined as mildly smaller or slower writing or difficulty in completing a writing task from baseline, but most words are legible; and
a second grade defined as moderate to severely smaller or slower writing or difficulty in completing task from baseline, and most words are illegible.

4. The system of claim 1, comprising grading criteria for gait impairment, that defines the symptoms of at least one adverse event selected from the group consisting of: Parkinsonian gait, gait disturbance, gait inability, and posture abnormal.

5. The system of claim 4, wherein the grading criteria for gait impairment defines the symptoms of at least four grades of severity of the adverse event, comprising:
a first grade defined as a slower walk that requires no assistance, and no festination or propulsion;
a second grade defined as walking with difficulty that requires little assistance;
a third grade defined as a severe gait disturbance that requires assistance; and
a fourth grade defined as an inability to walk even with assistance.

6. The system of claim 1, comprising grading criteria for other movement abnormalities of extremities, that defines the symptoms of at least one adverse event selected from the group consisting of: tremor, muscle rigidity, cogwheel rigidity, restlessness, akathisia, bradykinesia, hypokinesia, coordination abnormal, ataxia, and balance disorder.

7. The system of claim 6, wherein the grading criteria for other movement abnormalities of extremities grading criteria defines the symptoms of at least three grades of severity of the adverse event, comprising:
a first grade defined as mild symptoms of tremors at rest, rigidity, slowing of movements, restlessness, impaired coordination, or impaired balance;
a second grade defined as moderate symptoms of tremors at rest, rigidity, slowing of movements, restlessness, impaired coordination, or impaired balance; and
a third grade defined as severe symptoms of tremors at rest, rigidity, slowing of movements, restlessness, impaired coordination, or impaired balance.

8. The system of claim 1, comprising grading criteria for impairment in the ability to perform self-care activities of daily living, that defines the symptoms of at least one adverse event selected from the group consisting of: impaired self-care, dysphagia, dyspraxia, and apraxia.

9. The system of claim 8, wherein the grading criteria for impairment in the ability to perform self-care activities of daily living defines the symptoms of at least four grades of severity of the adverse event, comprising:
a first grade defined as performing self-care activities of daily living somewhat slower than baseline, but no help needed for self-care;
a second grade defined as requiring occasional assistance with self-care activities of daily living;
a third grade defined as requiring considerable help to perform self-care activities of daily living, but can do some things alone; and
a fourth grade defined as disabled and requiring full care.

10. The system of claim 1, comprising grading criteria for impairment in expression or motivation, that defines the symptoms of at least one adverse event selected from the group consisting of: flat affect, reduced facial expression, amimia, apathy, indifference, communication disorder, slow speech, and dysarthria.

11. The system of claim 10, wherein the grading criteria for impairment in expression or motivation defines the symptoms of at least two grades of severity of the adverse event, comprising:
a first grade defined as mildly flat affect, reduced facial expression or emotional response, less communicative, slower or mumbled speech, or disinterest in non-routine activities; and
a second grade defined as moderate to severely flat affect, lack of facial expression or emotional response, few word answers, monotonic or slurred speech, or disinterest in routine activities.

12. The method of claim 1, wherein the medical treatment is an immune effector cell therapy.

13. The method of claim 1, wherein the medical treatment is chimeric antigen receptor T-cell therapy.

14. A system for assessing neurologic toxicity in a subject associated with a medical treatment, comprising:
a grading criteria for handwriting impairment that defines the symptoms of at least one adverse event selected from the group consisting of: micrographia, dysgraphia, and agraphia;
a grading criteria for gait impairment that defines the symptoms of at least one adverse event selected from the group consisting of: Parkinsonian gait, gait disturbance, gait inability, and posture abnormal;
a grading criteria for other movement abnormalities of extremities that defines the symptoms of at least one adverse event selected from the group consisting of: tremor, muscle rigidity, cogwheel rigidity, restlessness, akathisia, bradykinesia, hypokinesia, coordination abnormal, ataxia, and balance disorder;
a grading criteria for impairment in the ability to perform self-care activities of daily living that defines the symptoms of at least one adverse event selected from the group consisting of: impaired self-care, dysphagia, dyspraxia, and apraxia; and
a grading criteria for impairment in expression or motivation that defines the symptoms of at least one adverse event selected from the group consisting of: flat affect, reduced facial expression, amimia, apathy, indifference, communication disorder, slow speech, and dysarthria;
wherein the grading criteria are stored in a non-transitory computer-readable medium.

15. The system of claim 14, wherein:
the grading criteria for handwriting impairment defines the symptoms of at least two grades of severity of the adverse event, comprising:
a first grade defined as mildly smaller or slower writing or difficulty in completing a writing task from baseline, but most words are legible; and
a second grade defined as moderate to severely smaller or slower writing or difficulty in completing task from baseline, and most words are illegible;
the grading criteria for gait impairment defines the symptoms of at least four grades of severity of the adverse event, comprising:
a first grade defined as a slower walk that requires no assistance, and no festination or propulsion;
a second grade defined as walking with difficulty that requires little assistance;
a third grade defined as a severe gait disturbance that requires assistance; and
a fourth grade defined as an inability to walk even with assistance;
the grading criteria for other movement abnormalities of extremities defines the symptoms of at least three grades of severity of the adverse event, comprising:
a first grade defined as mild symptoms of tremors at rest, rigidity, slowing of movements, restlessness, impaired coordination, or impaired balance;
a second grade defined as moderate symptoms of tremors at rest, rigidity, slowing of movements, restlessness, impaired coordination, or impaired balance; and
a third grade defined as severe symptoms of tremors at rest, rigidity, slowing of movements, restlessness, impaired coordination, or impaired balance;
the grading criteria for impairment in the ability to perform self-care activities of daily living defines the symptoms of at least four grades of severity of the adverse event, comprising:
a first grade defined as performing self-care activities of daily living somewhat slower than baseline, but no help needed for self-care;
a second grade defined as requiring occasional assistance with self-care activities of daily living;
a third grade defined as requiring considerable help to perform self-care activities of daily living, but can do some things alone; and
a fourth grade defined as disabled and requiring full care; and
the grading criteria for impairment in expression or motivation defines the symptoms of at least two grades of severity of the adverse event, comprising:
a first grade defined as mildly flat affect, reduced facial expression or emotional response, less communicative, slower or mumbled speech, or disinterest in non-routine activities; and
a second grade defined as moderate to severely flat affect, lack of facial expression or emotional response, few word answers, monotonic or slurred speech, or disinterest in routine activities.

16. The system of claim 14, wherein the medical treatment is an immune effector cell therapy.

17. The system of claim 14, wherein the medical treatment is chimeric antigen receptor T-cell therapy.

18. A method of assessing neurologic toxicity in a subject associated with a medical treatment, comprising the step of determining a toxicity grade according to at least one grading criteria selected from the group consisting of: a grading criteria for handwriting impairment, a grading criteria for gait impairment; a grading criteria for other movement abnormalities of extremities; a grading criteria for impairment in the ability to perform self-care activities of daily living; and a grading criteria for impairment in expression or motivation.

19. The method of claim 18, wherein:
the grading criteria for handwriting impairment defines the symptoms of at least one adverse event selected from the group consisting of: micrographia, dysgraphia, and agraphia;
the grading criteria for gait impairment defines the symptoms of at least one adverse event selected from the group consisting of: Parkinsonian gait, gait disturbance, gait inability, and posture abnormal;
the grading criteria for other movement abnormalities of extremities defines the symptoms of at least one adverse event selected from the group consisting of: tremor, muscle rigidity, cogwheel rigidity, restlessness, akathisia, bradykinesia, hypokinesia, coordination abnormal, ataxia, and balance disorder;
the grading criteria for impairment in the ability to perform self-care activities of daily living defines the symptoms of at least one adverse event selected from the group consisting of: impaired self-care, dysphagia, dyspraxia, and apraxia; and
the grading criteria for impairment in expression or motivation defines the symptoms of at least one adverse event selected from the group consisting of: flat affect, reduced facial expression, amimia, apathy, indifference, communication disorder, slow speech, and dysarthria.

20. The method of claim 19, wherein:
the grading criteria for handwriting impairment defines the symptoms of at least two grades of severity of the adverse event, comprising:
a first grade defined as mildly smaller or slower writing or difficulty in completing a writing task from baseline, but most words are legible; and
a second grade defined as moderate to severely smaller or slower writing or difficulty in completing task from baseline, and most words are illegible;
the grading criteria for gait impairment defines the symptoms of at least four grades of severity of the adverse event, comprising:
a first grade defined as a slower walk that requires no assistance, and no festination or propulsion;
a second grade defined as walking with difficulty that requires little assistance;
a third grade defined as a severe gait disturbance that requires assistance; and
a fourth grade defined as an inability to walk even with assistance;
the grading criteria for other movement abnormalities of extremities defines the symptoms of at least three grades of severity of the adverse event, comprising:
a first grade defined as mild symptoms of tremors at rest, rigidity, slowing of movements, restlessness, impaired coordination, or impaired balance;
a second grade defined as moderate symptoms of tremors at rest, rigidity, slowing of movements, restlessness, impaired coordination, or impaired balance; and
a third grade defined as severe symptoms of tremors at rest, rigidity, slowing of movements, restlessness, impaired coordination, or impaired balance;
the grading criteria for impairment in the ability to perform self-care activities of daily living defines the symptoms of at least four grades of severity of the adverse event, comprising:
a first grade defined as performing self-care activities of daily living somewhat slower than baseline, but no help needed for self-care;
a second grade defined as requiring occasional assistance with self-care activities of daily living;
a third grade defined as requiring considerable help to perform self-care activities of daily living, but can do some things alone; and
a fourth grade defined as disabled and requiring full care; and
the grading criteria for impairment in expression or motivation defines the symptoms of at least two grades of severity of the adverse event, comprising:
a first grade defined as mildly flat affect, reduced facial expression or emotional response, less communicative, slower or mumbled speech, or disinterest in non-routine activities; and
a second grade defined as moderate to severely flat affect, lack of facial expression or emotional response, few word answers, monotonic or slurred speech, or disinterest in routine activities.

21. The method of claim 18, wherein toxicity grades are determined according to at least two grading criteria, and further comprising the step of determining an overall toxicity grade as the toxicity grade of the most severely impaired grading criteria.

22. The method of claim 18, wherein the medical treatment is an immune effector cell therapy.

23. The method of claim 18, wherein the medical treatment is chimeric antigen receptor T-cell therapy.
